(19) United States
(12) Patent Application Publication
(10) Pub. No.: US 2003/0161819 A1
(43) Pub. Date: Aug. 28, 2003

(54) Reversible immortalization

(76) Inventors: Jan-Heiner Kupper, Kusterdingen (DE); Reinhard Kandolf, Hechingen (DE); Anne Kuhn, Tubingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/257,687

(22) PCT Filed: Mar. 15, 2001
(86) PCT No.: PCT/EP01/02967

(30) Foreign Application Priority Data
Apr. 17, 2000 (DE) 100.19.195.9

(51) Int. Cl. C07H 21/04; A61K 48/00; C12N 5/08; C12N 15/86

(52) U.S. Cl. 424/93.21; 435/456; 435/366; 536/23.2

(57) ABSTRACT

A gene complex for reversibly immortalizing cells contains an immortalizing gene region, which possesses at least a resistance gene, an immortalizing gene and, preferably, a suicide gene, and also two sequences which flank the gene region and which function as recognition sites for homologous intramolecular recombination, and at least one promoter located upstream of the gene region (FIG. 1). A gene complex for immunomodulating cells contains a first immunomodulating gene region, whose expression inhibits the function of MHC I molecules, a second immunomodulating gene region, whose expression leads to the inactivation of natural killer cells, and a resistance gene. A method for obtaining cells involves preparing organ-related cells which are immortalized by transferring the first gene complex and immunomodulated by transferring the second gene complex. After the immortalized cells have been expanded, the immortalization is reversed.

The present invention is concerned with methods for obtaining cells which can, for example, be transplanted into an organ. In a general manner, the present invention relates to degenerative diseases which have in common the destruction of defined cell populations, and also to transplants and drugs for treating such degenerative diseases.

Chronically degenerative diseases, which are difficult to treat or cannot be treated at all, are on the increase in industrial countries, particularly as a result of the changing age pyramid.

These diseases include, inter alia, cardiac muscle diseases, neurodegenerative diseases, bone diseases and liver diseases.

The severity of these diseases, and their increasing frequency in the ageing population, are associated with medical treatment which is becoming ever more expensive and with high consequential costs to the economy. This applies, in particular, to heart muscle diseases which can arise as a consequence of stenoses of the coronary blood vessels, of chronic cardiac muscle inflammation or of mechanical overload.

On the one hand, these diseases can lead to an acute myocardial infarction, which takes a fatal course in ⅓ of cases and is frequently heralded for many years previously by attacks of angina pectoris. The myocardial infarction leads to a massive necrotic or apoptotic destruction of contractile cardiac muscle cells. The area affected becomes scarred due to the multiplication of connective tissue cells and the deposition of extracellular matrix; however, cardiac muscle cells are not regenerated.

Another important complication is what is termed congestive heart failure, which is due to hypertrophy of the heart. While this hypertrophy initially represents a physiologically appropriate reaction to persistently increased stress, it leads, from a critical size onward, once again to a decrease in performance. Further enlargement beyond this critical point leads inevitably to heart failure. The only therapy for cardiac hypertrophy which is currently possible is heart transplantation.

It is known that cardiac hypertrophy is based on an expansion of individual cell types and on a remolding of contractile heart muscle tissue with connective tissue. The extremely differentiated cardiac muscle cells have lost their ability to regenerate by cell division. Various biochemical and mechanical stimuli even induce programmed cell death (apoptosis) in cardiac muscle cells.

Taken overall, both decompensated cardiac hypertrophy and myocardial infarction are characterized by a large loss of contractile cardiac muscle cells.

The loss of defined cell types can also determine the course of the affection in the case of neurodegenerative diseases. Thus, in the case of Parkinson's disease, which is the most frequent neurological disease in advanced age, there is a continuous decrease in the dopamine-producing cells in the substantia nigra. It is very probable that this decrease is also due to apoptotic cell death.

It has been possible to demonstrate that selective transplantation of fetal dopaminergic cells into the substantia nigra can very substantially improve the most severe clinical manifestations of Parkinson's disease.

Age-related osteoporosis, a progressive skeletal disease in the female population, in particular, is characterized by the loss of bone substance, with this loss being preceded by a decline in the number and activity of bone-forming cells (osteoblasts and osteocytes).

Liver damage develops progressively as a result of alcohol abuse or chronic inflammations or due to a metabolic cause or as a consequence of cardiovascular diseases and is characterized by the loss of physiologically active liver parenchymal cells (hepatocytes).

There is currently no curative therapy for any of these diseases which have been mentioned in this regard solely by way of example.

While the drugs which are prescribed for cardiac muscle diseases can exert a positive influence on cardiac functions such as contractility and conduction, they are unable to replace any heart muscle tissue which has been lost. In addition, drugs frequently only act symptomatically by, for example, withdrawing tissue water in association with portal hypertension following liver cirrhosis, or by alleviating pain in association with osteoporosis.

In the case of advanced cardiac muscle diseases or in the case of decompensated liver cirrhosis, organ transplantation is the only remaining option. However, this is limited by declining numbers of donor organs and is, furthermore, associated with high treatment costs. In addition to this, organ transplantation is associated with the inherent problem of tissue rejection, which means that the patients have to be treated with immunosuppressive agents for the remainder of their lives. The most serious complications which arise in this connection are the opportunistic infections with viruses, bacteria and fungi, which infections not infrequently take a fatal course.

In addition to this, there is not even the option of organ transplantation in the case of neurodegenerative diseases such as Parkinson's disease. However, in some cases of this disease, attempts to transplant dopaminergic cells from the brains of aborted fetuses have already met with a significant degree of success.

There are already isolated indications in the literature that the degenerative diseases which have in this respect been mentioned by way of example can be treated by transplanting cells. Kobayashi et al., “Prevention of acute liver failure in rats with reversibly immortalized human hepatocytes”, Science, Volume 287, pages 1258-1262, describe a retroviral vector which can be used for infecting primary human hepatocytes. The hepatocytes, which were replicated in vitro, were injected into the spleen of hepatectomized rats and it was possible to demonstrate that important liver functions were supported after the cells had been injected. The method proposed in this publication is intended to bridge the time until transplantation or until the liver regenerates spontaneously. In principle, this publication demonstrates that cell transplantation can be used to produce clinically desirable effects.

The retroviral vector which is used in the known method contains a gene region possessing a resistance gene, a suicide gene and a transformation gene, which region is flanked by two LoxP sites. The downstream LoxP site is followed by another resistance gene which, however, lacks an initiation codon such that this second resistance gene cannot be translated. The transformation gene, in this case the SV40 tumor antigen, drives the resting hepatocytes to proliferate once again, with the first resistance gene giving rise to resistance to hygromycin and the suicide gene giving rise to sensitivity toward ganciclovir. In this way, it is possible to select for transformation.

One of the cell lines which was selected in this way was evidently immortal and could be expanded in an appropriate medium. After the expansion, the cells were transduced with a replication-incompetent recombinant adenovirus which expressed the Cre recombinase. The Cre recombinase excised the gene region located between the two LoxP sites such that the SV40 T-Ag oncogene was no longer expressed; for information on the Cre-Lox system, see, for example, Rajewsky et al., “Conditional gene targeting”, J. Clin. Invest., Volume 98, pages 600-603. The excision with the Cre recombinase resulted in an intramolecular recombination such that the resistance gene located outside the gene region flanked by the LoxP sites now came to be located immediately downstream of a start codon and was consequently able to mediate a resistance. In this way, it was possible to verify the success of the excision by, in this case, selecting for resistance to G418 (neomycin resistance gene).

While said publication describes this process as being reversible immortalization, it is, according to the findings of the inventors of the present application, a reversible transformation which, in the case of one clone, has led to immortal cells as a result of spontaneous mutation. Thus, it is known that the rule when transforming cells with SV40 T-Ag is that it is only possible to produce an “extended life span”; in this regard, see, for example, Chiu and Harley, “Replicative senescence and cell immortality: the role of telomeres and telomerase”, Proc. Soc. Exp. Biol. Med., Volume 214, pages 99-106. Chiu and Harley provide a brief overview of the telomere-hypothesis, which is based on telomere length serving as a systematic clock for regulating the replicative life-span of cells. These authors report that telomerase expression stabilizes telomere length and renders continuous replication, or cell immortality, possible. They also describe the therapeutic possibilities which are linked to this hypothesis and discuss whether it is possible to increase the replicative potential of cells by activating telomerase expression in vivo or ex vivo. They furthermore report that telomerase activity, which has been elicited by a spontaneous mutation, is present in many tumor cells. Based on the article by Chiu and Harley, it can be assumed that the immortal cell line reported by Kobayashi et al., loc. cit., arose as the result of a spontaneous mutation following transformation with the oncogene. However, this means that the method described by Kobayashi et al., loc. cit., is not reproducible and can consequently not be usefully employed commercially.

When human primary cells are cultured, they divide a further 20-60 times, depending on the age of the donor, and then go into senescence. The telomere loss which occurs at each division induces a cessation in cell division, something which can be circumvented by transforming the cells with an oncogene. These cells can then continue to divide beyond this first crisis. However, a second crisis then arises at some point since the telomere loss which occurs at each cell division leads to genetic instability. This second crisis is fatal for almost all the cells which have been transformed with a tumor antigen. However, in fewer than 10⁻⁶ of cases, spontaneous mutation results in activation of the telomerase, meaning that the telomere loss can be compensated for from that time onward. This is termed spontaneous immortalization, as must also have taken place in the case of Kobayashi et al. loc. cit. This telomerase activation cannot be selectively switched off once again, either, which means that, even after the tumor antigen has been excised, the risk remains that these cells may degenerate into cancer cells as the result of a further mutational event which once again brings about induction of growth.

The method which is described by Kobayashi et al. in this regard consequently suffers from a whole series of disadvantages. In the first place, the success of the method depends on the telomerase being spontaneously activated. However, this means that the method is not reproducible and can consequently only be used commercially to a very limited extent. In the second place, it is not possible to switch the telomerase activity off again selectively, which means that the cells can degenerate into cancer cells even after the tumor antigen has been excised. A further disadvantage is that, even after the excision, a part of the retroviral vector remains active in the expanded cells and expresses the resistance gene which is used for selecting for successful excision. However, this additional genetic material stands in the way of the cells which have thus been treated being transplanted into human organs.

While Kobayashi et al. are concerned with preparing transplantable cells from terminally differentiated parent cells, it is also already known to transplant autologous bone marrow stroma cells into the heart for the purpose of improving cardiac function; see Tomita et al., “Autologous transplantation of bone marrow cells improves damaged heart function”, Circulation, Volume 100, pages II247-II256. The bone marrow cells were cultured in the added presence of the differentiation substance 5′-azacytidine, resulting in the bone marrow cells differentiating into cardiomyogenic cells.

However, because of the limited replicative capacity of the autologous bone marrow stroma cells, it is not possible to reproduce the quantity of regenerative cells which is required in humans in this way. Since cardiac diseases are diseases of old age, most autologous donors are also relatively old, which means that the autologous bone marrow cells which are withdrawn from the patient are perhaps able to divide a further twenty times. Consequently, it is theoretically only possible to still prepare approx. 10⁶ cells from a cell, corresponding to less than 0.02% of the left ventricle. The method described by Tomita et al., loc. cit., is consequently not suitable for clinical applications.

Makino et al., “Cardiomyocytes can be generated from marrow stromal cells in vitro”, J. Clin. Invest., Volume 103, pages 697-705, are also concerned with differentiating bone marrow cells into cardiac muscle cells by adding 5′-azacytidine. An immortalized cell line was obtained by frequently subculturing the stroma cells for a period of more than four months, with this immortalized cell line then being used as the starting material for the differentiation into cardiac muscle cells. This method suffers from the same disadvantages as the method described by Kobayashi et al., loc. cit.; it can neither be reproduced nor tolerated clinically.

In view of the above, it is an object of the present invention to provide in a cost-effective manner immunologically and clinically harmless cells which can be used, for example, for regenerating tissue locally.

According to the invention, this object is achieved by means of a method for obtaining cells, comprising the steps of: preparing organ-related cells, immortalizing the organ-related cells, expanding the immortalized cells and reversing the immortalization of the expanded cells. In this connection, the organ-related cells can be multipotent stem cells, preferably mesenchymal stroma cells or else resting, terminally differentiated parent cells of the organ.

As a result of being reversibly immortalized, the cells which have been prepared in this way are clinically harmless. In addition, the cells can be prepared in unlimited number.

When multipotent stem cells are used as the organ-related cells, the immortalized stem cells are expanded in the added presence of at least one differentiation substance which promotes differentiation of the stem cells into organ-specific cells.

On the other hand, if terminally differentiated parent cells are used, these cells are additionally transformed in connection with the immortalization so as to ensure that they can be expanded.

The cells which have been prepared in this way can be used, for example, to carry out a transplantation in a cardiac infarction area, thereby simultaneously and substantially reducing the risk of a congestive heart failure and of a secondary, fatal cardiac infarction. The method is also suitable for obtaining regenerative bone cells and cartilage cells, which cells can be used in connection with bone and cartilage traumas and in connection with chronic bone degeneration (osteoporosis). The method can also be used to prepare liver parenchymal cells for liver regeneration and dopaminergic cells for treating Parkinson's disease.

The method according to the invention makes it possible to produce any desired quantities of primary cells for the purpose of preparing tissue extracorporeally. Endothelial cells or smooth muscle cells which have been produced in accordance with the novel method can be used to colonize a matrix, preferably a biomatrix, for example made of collagen or fibronectin, for the purpose of generating heart or venous valves.

When muscle cells, preferably cardiac muscle cells, and also bone cells, are, for example, being prepared, it is preferred if a differentiation substance, which is selected from the group: dexamethasone, 5′-azacytidine, trichostatin A, all-trans retinoic acid and amphotericin B, is added when the immortalized stem cells are being expanded. In this connection, it is particularly preferred if at least two, preferably four, of these differentiation substances are used in association with the expansion.

Although differentiation of stem cells into cardiac muscle cells is induced by adding 5′-azacytidine, the differentiation can be improved by adding at least one further differentiation substance. A combination of 5′-azacytidine and trichostatin A, which, according to the findings of the inventors of the present application acts synergistically, is particularly suitable in this connection. The differentiation can be further optimized by additionally adding all-trans retinoic acid and amphotericin B.

In addition to the synergistic effect to be obtained from combining several differentiation substances, a further advantage is the fact that the mutagenic effect which the inventors have found 5′-azacytidine to possess is substantially reduced or even abolished.

In this way, it is possible to safely use the cardiac muscle cells, which have thus been obtained from stem cells, for clinical purposes.

The reversibly immortalized stem cells are differentiated into bone cells and cartilage cells by adding the differentiation substance dexamethasone. In this case, too, it is possible to achieve a synergistic effect by additionally adding the differentiation substances 5′-azacytidine, trichostatin A, all-trans retinoic acid and amphotericin B.

With regard to the differentiation substance dexamethasone, mention should also be made of the fact that Conget and Minguell “Phenotypical and functional properties of human bone marrow mesenchymal progenitor cells”, J. Cell. Physiol., Volume 181, pages 67-73, have already reported that osteogenic cells can be obtained from mesenchymal stroma cells by treating with dexamethasone.

The organ-related cells which can be used in this connection can be either autologous cells or allogenic cells.

While the advantage of the autologous cells lies in the immunotolerance, the allogenic cells have the advantage that they are more or less at any time available in unlimited number. In the case of the allogenic cells, however, an immunotolerance is generated, according to the invention, in order to reduce the host-versus-graft reaction (HVGR).

It is then consequently possible, for the purpose of treating a patient, to initially use transplantable cells which have been prepared from allogenic cells while further transplantable cells are being prepared in parallel from autologous cells derived from the patient. When sufficient autologous transplantable cells are available, it is then only these cells which are transplanted, thereby ensuring that immunotolerance no longer constitutes any problem.

According to the invention, a gene complex containing an immortalizing gene region, which possesses at least a resistance gene, an immortalizing gene and, preferably, a suicide gene, containing two sequences which flank the gene region and which function as recognition sites for homologous intramolecular recombination, and containing at least one promoter which is located upstream of the gene region, is used for reversibly immortalizing the cells.

When this gene complex is introduced into an organ-related cell, it is possible to initially use the resistance gene to select for successful transfer.

As a result of the presence of the immortalizing gene, which is preferably the telomerase gene, telomere loss is now avoided during the expansion, meaning that the cells are able to replicate without limit provided that the organ-related cells are stem cells which are capable of proliferation.

When, on the other hand, the organ-related cells employed are terminally differentiated parent cells, the gene complex then additionally contains a transformation gene which is preferably the SV40 tumor antigen, that is an oncogene. In this way, it is also possible to immortalize the resting cells and use them for preparing transplantable cells.

After expansion has taken place, the gene region which contains the resistance gene, the immortalizing gene and the suicide gene is excised from the gene complex by homologous, intromolecular recombination, thereby ensuring that the immortalization is reliably abolished. As a consequence, the risk of the cells which have been prepared in this way degenerating into cancer cells after they have been transplanted is no greater than is usually the case.

The flanking sequences in this connection are preferably LoxP sites, with Cre recombinase being used for the intramolecular recombination. In this connection, the immortalization of the expanded cells can be reversed at any desired point in time by infecting the cells with, for example, a recombinant virus, for example a recombinant adenovirus, which expresses the Cre recombinase, or by administering the Cre recombinase as a recombinant fusion protein which can enter cells, for example as a fusion protein with the voyager protein VP22.

The suicide gene is preferably used in this connection for selecting for successful excision. The suicide gene is preferably the hepatitis simplex virus (HSV) thymidine kinase gene. After the immortalizing gene region has been excised, the suicide gene has also been removed from the transferred gene complex, which means that these cells are no longer sensitive to ganciclovir. However, the sensitivity toward ganciclovir is still present in the cells in which excision has not taken place, resulting in these cells being killed.

A great advantage of the method which has been described in this regard is to be seen in the fact that, after the immortalization has been reversed, the gene complex no longer contains any expressible genes, which means that the transplantation of these cells is completely harmless from the clinical point of view.

When allogenic cells are used as organ-specific cells, immunotolerance is elicited in the allogenic cells by transferring a gene complex into these cells. In this connection, the gene complex for immunomodulating cells comprises a first immunomodulating gene region, whose expression inhibits the function of MHC I molecules on the cells, and a second immunomodulating gene region, whose expression leads to the inactivation of natural killer cells (NK cells). The gene complex for the immunomodulation further comprises a resistance gene which is used for selecting for successful transfer.

The first immunomodulating gene region in this connection contains a gene which is selected from the group: CMV genes US2 and US11; HSV gene ICP47; CMV genes US6 and US3; adenovirus genes E3-19K and E6; HIV NEF gene and gene for a recombinant single-chain antibody for blockading the presentation of MHC I on the cell surface.

According to the invention, the second immunomodulating gene region contains the CMV gene UL18 or a gene for a recombinant single-chain antibody which anchors in the membrane of the cell and repulses natural killer cells.

The immunomodulation which has been brought about in this way is used to engender immunotolerance in the immortalized allogenic, organ-specific cells, which immunotolerance enables the cells to be transplanted without risk into allogenic recipients.

The immunotolerance is achieved, in the first place, by blockading the appearance of MHC I molecules on the cell surface. This keeps the recipient's cytotoxic T lymphocytes from lysing the exogenous donor cells. In this connection, by expressing the CMV genes US2 and US11 it is possible to prevent MHC I molecules from populating the cell surface.

Miller and Sedmak, “Viral effects on antigen processing”, Curr. Opin. Immunol., Volume 11, pages 94-99, were able to show that these gene products of the human cytomegalovirus (CMV) bring about extensive degradation of intracellular MHC I molecules.

Since a decrease, or complete blockade, of MHC I presentation on cells leads to activation of natural killer cells, the activity of these natural killer cells is inhibited by expressing the CMV gene UL18.

The inactivation of MHC I can also be effected using other viral genes, for example the HSV gene ICP47, the CMV genes US6 and US3, the adenovirus genes E3-19K and E6, or the HIV NEF gene. Furthermore, direct knock-out of an MHC I component, e.g. the beta-2-microglobulin gene, is also possible. It is furthermore also possible to express intracellularly a recombinant single-chain antibody which blocks MHC presentation on the cell surface.

Natural killer cells can also be inactivated using a monoclonal antibody which recognizes what are termed the inhibitory receptors on the natural killer cells and blocks NK-mediated cell lysis. According to the invention, this monoclonal antibody is also anchored, as a recombinant single-chain antibody, in the membrane of the allogenic donor cell and can thereby fend off attacking NK cells.

Cells which have been prepared using the novel method, and, where appropriate, using the novel gene complexes, are likewise part of the subject matter of the present invention. According to the invention, these cells can be used for preparing a transplant for regenerating an organ or for producing a drug for treating chronic diseases.

Against this background the present invention likewise relates to a drug which comprises a therapeutically effective quantity of the cells which have been prepared in accordance with the invention and to a transplant which contains these cells.

The present invention furthermore relates to the use of the cells for regenerating an organ.

Further, the invention also relates to a plasmid, to a viral vector or to a kit which contains the gene complex for the reversible immortalization and/or the gene complex for immunomodulating cells.

The plasmid according to the invention and/or the viral vector according to the invention is/are used for transferring the gene complexes into the organ-specific cells in order to immortalize, where appropriate transform, and where appropriate exert an immunomodulating effect on, these cells.

In addition to the gene complexes, the kit according to the invention can contain the other substances and materials required, for example recombinant adenoviruses encoding the Cre recombinase.

The kit can then be used to reversibly immortalize and expand allogenic or autologous donor cells before the latter are then transplanted for the purpose of organ regeneration.

Other advantages ensue from the description and the attached drawing.

It will be understood that the features which are mentioned above, and those which are still to be explained below, can be used not only in the combinations which are in each case indicated but also in other combinations, or on their own, without departing from the scope of the present invention.

The invention is now explained with the aid of embodiments and the enclosed drawing, in which:

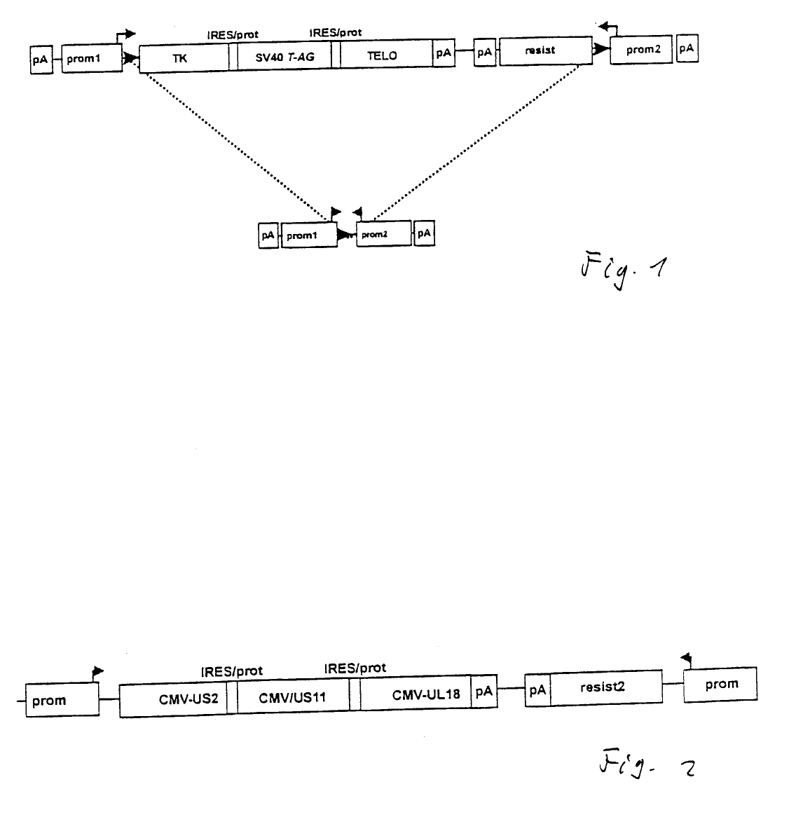

FIG. 1 shows a diagram of a gene complex for reversibly immortalizing cells; and

FIG. 2 shows a diagram of a gene complex for immunomodulating cells.

### EXAMPLE 1

### Providing Organ-Related Cells

The organ-related cells which are used can be multipotent stem cells which still have to be differentiated into organ-specific cells in association with the expansion or else parent cells of the given organ which have already been differentiated.

In addition, it is necessary to distinguish between autologous cells from the given patient and allogenic cells from a donor. While autologous cells can be used without further immunomodulatory treatment, allogenic cells are stably transfected with immunomodulatory genes. The advantage of allogenic cells is that these cells can be prepared in large numbers and can be used immediately for many recipients, with the risk of a host-versus-graft reaction (HVGR) being very low due to the immunomodulatory treatment (see Example 2). An advantage of autologous cells is that there is no HVGR risk.

The stem cells employed are bone marrow mesenchymal stroma cells. These cells are able to differentiate into osteoblasts, myoblasts, adipocytes and other cell types. In hospitals, bone marrow is routinely obtained under surgical conditions for allogenic bone marrow transplantation. However, it is only the hematopoietic stem cells which are required in this connection, whereas the mesenchymal stem cells, which are of interest in the present case, are obtained as a by-product.

On the other hand, mesenchymal stem cells can also be isolated from peripheral blood.

The stem cells which are obtained in this way are sown in conventional cell culture dishes and cultured in alpha MEM or IDEM medium containing 10% fetal calf serum and antibiotics such as penicillin, streptomycin or amphotericin B.

Liver hepatocytes are established directly as a primary culture. Dopaminergic parent cells are removed within the context of an organ donation. Cardiac muscle cells can be obtained for the immortalization both as bone marrow stem cells and as parent cells within the context of a heart muscle biopsy.

### EXAMPLE 2

### Immortalizing, Transforming and Immunomodulating

The organ-related cells which have been obtained in this way are now reversibly immortalized, with the terminally differentiated parent cells also having to be transformed. An immunomodulation is also additionally required in the case of allogenic organ-related cells.

The gene complex depicted in FIG. 1 is used for reversibly immortalizing the organ-related cells. On the other hand, the gene complex depicted in FIG. 2 is used for immunomodulating allogenic donor cells.

Both the gene complexes can be introduced into the organ-related target cells by means of plasmid transfection or by means of viral transduction. The respective resistance gene can be used for selecting for successful transfer of the gene complexes.

The resistance gene in the gene complex shown in FIG. 1 is, for example, the neomycin gene, which mediates resistance to G418. The resistance gene in FIG. 2 is, for example, the hygromycin gene, which mediates resistance to hygromycin.

The gene complex in FIG. 1 contains the SV40 large tumor antigen as the transforming gene and the telomerase gene as the immortalizing gene. In this connection, the transforming gene is only required for resting, terminally differentiated parent cells; the immortalizing gene, encoding the telomerase, is sufficient for proliferating stem cells.

In addition, the gene complex depicted in FIG. 1 contains a suicide gene, namely the thymidine kinase gene, which mediates sensitivity to ganciclovir.

The immortalizing gene region, comprising the suicide gene TK, the transforming gene SV40T-Ag and the telomerase gene TELO, and also the first resistance gene resist, is flanked by two LoxP sites. The bacteriophage P1 enzyme Cre recombinase can be used to bring about homologous recombination at two identical LoxP sequences. A LoxP site is a 34 base pair DNA sequence which is composed of two 13 base pair inverted repeats which are separated by an 8 base pair nonpalindromic sequence. When two LoxP sites are positioned in the same orientation on a linear DNA molecule, the Cre recombinase brings about an intramolecular recombination which leads to excision of the sequence located between the two LoxP sites. This is highly specific and very efficient since the excised DNA is removed from the equilibrium by degradation.

The expression of the tandemly arranged genes can be brought about by gene fusion, by internal translation using an IRES (internal ribosomal entry site) or by internal proteolysis (prot). In the case of the last-mentioned possibility, a protease, which excises itself in cis and consequently separates the individual gene functions, is located between the genes. The FMDV (foot and mouth disease virus) 2A protease can be used as the internal protease.

After the gene complex depicted in FIG. 1 has been successfully transferred into the organ-related cells described in Example 1, these cells can then be expanded at will. If the organ-related cells are multipotent stem cells, the expansion takes place in the added presence of at least one differentiation substance which promotes differentiation of the stem cells into organ-specific cells, as described below in Example 3.

When the organ-related cells are allogenic cells obtained from a donor, the gene complex depicted in FIG. 2 also has to be transferred, in addition to the gene complex depicted in FIG. 1, in order to achieve immune tolerance.

This takes place by expressing the CMV genes US2 and US11, which prevent the cell surfaces of the organ-related cells from being populated with MHC I molecules. While decreasing or completely blockading the presentation of MHC I on the one hand prevents the exogenous donor cells from being lysed by cytotoxic T lymphocytes, it automatically leads, on the other hand, to natural killer cells being activated.

According to the invention, the activity of the natural killer cells is now inhibited, by means of a mechanism which is still not precisely understood, by expressing the CMV gene UL18.

In FIGS. 1 and 2, pA denotes a poly(adenylation) signal and prom denotes a promoter. resist2 is a resistance gene which is different from resist.

The MHC I can also be inactivated using other viral genes, e.g. the HSV gene ICP47, the CMV genes US6 and US3, the adenovirus genes E3-19K and E6 or the HIV NEF gene, by direct knock-out of an MHC I component, such as the beta-2-microglobulin gene, or by the intercellular expression of a recombinant single-chain antibody for the purpose of blockading the presentation of MHC I on the cell surface.

The natural killer cells can also be inhibited using a monoclonal antibody which recognizes what are termed the inhibitory receptors of the natural killer cells and blocks the cell lysis which is mediated by natural killer cells. This monoclonal antibody can also, as a recombinant single-chain antibody, be anchored in the membrane of the allogenic donor cell and thereby fend off attacking natural killer cells.

The preparation of a membrane-located single-chain antibody is described in principle in Einfeld et al., “Construction of a pseudoreceptor that mediates transduction by adenoviruses expressing a ligand in fiber or penton base”, J. Virol., Volume 73, pages 9130-9136.

A hybridoma which produces a monoclonal antibody is prepared in a first step; see, for example, Immunobiologie [Immunobiology], 3rd edition, Janeway et al., Current Biology Limited & Churchill Livingstone & Garland Publishing Inc., 1997. The hybridoma is the fusion of a mortal antibody-producing cell from the spleen of an immunized mouse with an immortal myeloma cell.

The fused cells possess the properties of both parent cells, namely the ability to produce antibody and the ability to be immortal. The hybridoma cells are multiplied in a special selection medium (HAT medium) and analyzed with regard to expression of the antibody of interest.

The next step is that of cloning the gene segments which encode the variable regions of the light and heavy chains of the monoclonal antibody. The gene sequences which are crucial for recognizing the antigen are amplified by PCR using consensus primers within these variable regions. The gene sequence for the variable light chain is fused to the gene sequence for the heavy chain by way of a short linker which encodes approx. 15 amino acids. This gives rise to what is termed the single-chain Fv (fragment variable).

When expressed in cells, such a single-chain antibody can be inserted into the cell membrane using other signal sequences and anchoring sequences, thereby giving rise to the membrane-located single-chain antibody which is used in accordance with the invention.

However, instead of the recognition of a linear hemagglutinin epitope, as described by Einfeld et al., the antibody according to the invention recognizes an epitope on natural killer cells, thereby blocking NK-mediated cell lysis. The gene for this single-chain antibody can be contained in the gene complex depicted in FIG. 2 in place of the UL18 CMV gene.

According to the invention, single-chain antibodies can also be used in the sense of “intrabodies”, such that they recognize intracellular epitopes and thereby block the assembly or transport of MHC I molecules. The gene for such an antibody can also be present in the gene complex depicted in FIG. 2 and replace, for example, the CMV genes US2 and US1.

These single-chain antibodies are selected from the group: antibodies directed against TAP transporters (anti-TAPI and anti-TAPII), β2-microglobulin, calnexin, calreticulin and tapasin. The expression and activity of one or more of these intracellular single-chain antibodies prevent MHC I molecules from being presented on the surfaces of allogenic cells and thereby avoid the cells being lysed by cytotoxic T lymphocytes.

The direct knock-out of an MHC I component is based on a method which was originally described for embryonic stem cells, for the purpose of preparing transgenic mice. A general review of the “knocking-out” of genes is to be found in Koch-Brandt, “Gentransfer Prinzipien—Experimente—Anwendung bei Säugern” [Principles of gene transfer—experiments—use in mammals], Thieme Verlag, 1993, and also in Sedivy and Dutriaux, “Gene targeting and somatic cell genetics—a rebirth or a coming of age?”, Trends. Genet., Volume 15, pages 88-90.

In this method, a region which is homologous with the gene which is to be knocked-out is cloned into a plasmid, with a resistance gene being located within this homologous region and a suicide gene being located outside the homologous region. The plasmid is then transfected into a cell, with the homologous region being in rare cases integrated into the target gene.

The resistance gene which is present within the homologous region on the one hand makes it possible to select for integration events and on the other hand interrupts expression of the target gene on the allele concerned. Since the suicide gene is located outside the homologous region, this gene is only concomitantly integrated in association with an illegitimate recombination but not in association with a homologous recombination.

The suicide gene is used to select against cells in which an illegitimate recombination of the suicide gene has taken place. This method is consequently used to initially knock out one of the two alleles of a gene; in the case of the present invention, for example, the human gene for β2-microglobulin, which is an integral component of the MHC I complex. Elimination of the expression of β2-microglobulin consequently results in the complete absence of MHC I molecules on the cell surface and thereby prevents cell lysis which is mediated by cytotoxic T lymphocytes.

In order to knock out the second allele as well, the same strategy has to be pursued using a second resistance gene. This gene can integrate into the second allele by way of the identical homologous sequence. The two resistances and the suicide gene are now used to select for this result.

The preparation of β2-microglobulin knock-out mice has already been described by Koller and Smithies, “Inactivating the beta 2-microglobulin locus in mouse embryonic stem cells by homologous recombination”, PNAS, Volume 86, pages 8932-8935. There is no example in the literature for the case of human cells and the use of such cells, which are resistant to cytotoxic T lymphocytes, for preparing organ-related allogenic cells.

As well as knocking out components of the MHC I complex, the above-described approach can also be used to knock out one or both genes for the TAP transporter, with this likewise resulting in MHC I molecules no longer being presented on the cell surface. Mice which are knock-out for TAP1 are also known, see Behar et al., “Susceptibility of mice deficient in CD1D or TAP1 to infection with Mycobacterium tuberculosis”, J. Exp. Med., Volume 189, pages 1973-1980.

### EXAMPLE 3

### Expansion and Differentiation

Immortalized parent cells, which are already terminally differentiated, are expanded in a customary medium without further measures being required.

However, if the immortalized cells are bone marrow mesenchymal stem cells, it is necessary to differentiate them into the organ-specific sites by adding differentiation substances.

It is possible to differentiate mesenchymal stem cells into cardiomyogenic cells, for example, by treating them with 5′-azacytidine; Makino et al., loc. cit. Treating stem cells which possess the developmental potential of cardiac muscle cells with 5′-azacytidine induces differentiation processes as a result of demethylation. In this connection, the promoter is very probably activated by essential cardiac muscle differentiation genes which are still unknown.

However, according to the inventors' findings, 5′-azacytidine has a mutagenic potential. For this reason, the differentiation of stem cells into cardiac muscle cells is improved, according to the invention, by adding at least one further differentiation substance. The substance trichostatin A (TSA) is envisaged for this purpose. TSA inhibits histone deacetylation. This histone deacetylation is connected with transcriptional repression of CpG methylations.

CpG islands, that is regions containing several CpG dinucleotides, are to be chiefly found in promoters. The methylations can substantially inhibit the activity of a CpG-rich promoter. This occurs, for example, when 5′-azacytidine is incorporated into the DNA of replicating cells since no methylation as a result of cellular processes can take place at position 5 due to the aza group being at this position.

A combination of 5′-azacytidine and TSA can consequently act synergistically, as has already been demonstrated in tumor cells; see Cameron et al., “Synergy of demethylation and histone deacetylase inhibition in the re-expression of genes silenced in cancer”, Nat. Genet., Volume 21, pages 103-107.

According to the invention, this synergism is applied to the differentiation of stem cells into cardiac muscle cells. The differentiation is further optimized by additionally adding all-trans retinoic acid and amphotericin B. Retinoic acid is a differentiation substance which, in the myoblast cell line H9C2, favors a heart muscle phenotype over a skeletal muscle phenotype; see Menard et al., “Modulation of L-type calcium channel expression during retinoic acid-induced differentiation of H9C2 cardiac cells”, J. Biol. Chem., Volume 274, pages 29063-29070.

Amphotericin B is also able to exert a favorable influence on differentiation in the direction of cardiac muscle cells; see Phinney et al. “Plastic adherent stromal cells from the bone marrow of commonly used strains of inbred mice: variations in yield, growth, and differentiation”, J. Cell. Biochem., Volume 72, pages 570-585.

The advantage of using a combination of several differentiation substances is that this achieves synergistic effects which substantially reduce, or even abolish, the mutagenic effect of 5′-azacytidine. This is of crucial importance for the subsequent clinical use of stem-cell-derived cardiac muscle cells.

When the differentiation substance dexamethasone, Conget et al., loc. cit., is used on its own or in combination with the four above-described differentiation substances, it is possible to differentiate stem cells into bone cells and cartilage cells.

### EXAMPLE 4

### Reversing the Immortalization

In order to enable the cells which have been expanded, and, where appropriate, differentiated, as described in Example 3 to be subsequently transplanted, it is necessary for the immortalization to be abolished once again so as to ensure that the transplanted cells do not degenerate into tumor cells.

This is achieved by using the enzyme Cre recombinase to excise the gene region between the two LoxP sites from the gene complex depicted in FIG. 1. This can be done by infecting the cells with a recombinant adenovirus (Ad-Prom-Cre) which expresses the Cre recombinase. In this way, the immortalization can be reversed at any desired point in time.

The excised DNA sequences can no longer be expressed in the expanded cells, either, because the promoters are lacking since they remain in the homologously recombined gene complex as shown in FIG. 1. Nor can the promoters which are integrated in the cell DNA activate any adjacent cellular sequences in cis since the promoters are flanked by poly(adenylation) signals.

However, as a further safety measure, the TK suicide gene is also incorporated into the gene complex shown in FIG. 1. Cells whose gene complex shown in FIG. 1 is still intact express thymidine kinase and can be killed selectively by adding ganciclovir.

As an alternative to infecting cells with Ad-Prom-Cre, the Cre recombinase enzyme can also be administered as a fusion protein, for example as recombinant Cre-VP22. This fusion protein, which can enter cells, is added to the cell culture medium and, because of the fusion containing the voyager protein VP22, diffuses into the expanded cells.

### EXAMPLE 5

### Transplantation

After the immortalization has been reversed as described in Example 4, and after an appropriate quality control, conventional techniques are used to transplant the cells into the damaged organs, for example by injecting them into the organ using a syringe. This can take place repeatedly since material is available in any desired quantity due to the immortalization.

Without the immortalization, only from approx. 5×10⁸ to 1×10⁹ cells, and in the case of an elderly donor, possibly even only 1×10⁶ cells, could be derived from one stem cell. This would probably be too few for a regeneration. Furthermore, in the case of autologous transplantation, the patient has to wait until the cells have multiplied so as to achieve the requisite number of cells. By contrast, when the transplantation is allogenic, the desired number of cells can be provided at any time.

In cases of special need, it is appropriate to firstly carry out an allogenic transplantation and then subsequently to switch over to autologous transplantation.

1. A gene complex for reversibly immortalizing cells, containing an immortalizing gene region, which possesses at least a resistance gene, an immortalizing gene and, preferably, a suicide gene, two sequences which flank the immortalizing gene region and which function as recognition sites for homologous intramolecular recombination, and at least one promoter which is located upstream of the immortalizing gene region.

2. The gene complex of claim 1, characterized in that it additionally contains a transforming gene.

3. The gene complex of claim 1 or 2, characterized in that the suicide gene is the thymidine kinase gene.

4. The gene complex of claim 2 or 3, characterized in that the transforming gene is an oncogene, preferably the SV40 tumor antigen.

5. The gene complex of anyone of claims 1 to 4, characterized in that the immortalizing gene is the telomerase gene.

6. The gene complex of anyone of claims 1 to 5, characterized in that the flanking sequences are LoxP sites.

7. A gene complex for immunomodulating cells, containing a first immunomodulating gene region, whose expression inhibits the function of MHC I molecules on the cells, a second immunomodulating gene region, whose expression leads to the inactivation of natural killer cells, and a resistance gene.

8. The gene complex of claim 7, characterized in that the first immunomodulating gene region contains a gene which is selected from the group: CMV genes US2 and US11; HSV gene ICP47; CMV genes US6 and US3; adenovirus genes E3-19K and E6; HIV NEF gene and gene for a recombinant single-chain antibody for blockading the presentation of MHC I on the cell surface.

9. The gene complex of claim 7 or claim 8, characterized in that the second immunomodulating gene region contains the CMV gene UL18 or a gene for a recombinant single-chain antibody which becomes anchored in the membrane of the cell and fends off natural killer cells.

10. A method for obtaining cells, comprising the steps of:
preparing organ-related cells, immortalizing the organ-related cells, expanding the immortalized cells and reversing the immortalization of the expanded cells.

11. The method of claim 10, characterized in that the organ-related cells employed are multipotent stem cells, preferably bone marrow mesenchymal stroma cells.

12. The method of claim 11, characterized in that the immortalized stem cells are expanded in the added presence of at least one differentiation substance which promotes differentiation of the stem cells into organ-specific cells.

13. The method of claim 12, characterized in that the differentiation substance is selected from the group: dexamethasone, 5′-azacytidine, trichostatin A, all-trans retinoic acid and amphotericin B.

14. The method of claim 13, characterized in that a combination of at least two, preferably four, differentiation substances is used.

15. The method of claim 10, characterized in that the organ-related cells employed are resting, terminally differentiated parent cells of the organ.

16. The method of claim 15, characterized in that the parent cells are transformed in connection with the immortalization.

17. The method of anyone of claims 10 to 16, characterized in that the immortalization is effected by transferring the gene complex of anyone of claims 1 to 6 into organ-related cells.

18. The method of claim 17, characterized in that the resistance gene is used to select for successful transfer.

19. The method of anyone of claims 10 to 18, characterized in that the organ-related cells employed are autologous cells.

20. The method of anyone of claims 10 to 18, characterized in that the organ-related cells employed are allogenic cells.

21. The method of claim 20, characterized in that an immunotolerance is generated in the allogenic cells.

22. The method of claim 21, characterized in that the immunotolerance is elicited by transferring the gene complex as claimed in anyone of claims 7 to 9 into the allogenic cells.

23. The method of claim 22, characterized in that the resistance gene is used to select for successful transfer.

24. The method of claim 21, characterized in that an immunomodulation is brought about by a monoclonal antibody which recognizes the inhibitory receptors of natural killer cells and blocks the cell lysis which is mediated by natural killer cells.

25. The method of claim 21, characterized in that MHC I presentation on the cell surface is blocked by knocking out at least one gene in the allogenic cells, preferably encoding β2-microglobulin or encoding a TAP transporter.

26. The method of anyone of claims 17 to 25, characterized in that the immortalization is reversed by excising the immortalizing gene region from the gene complex present in the expanded cells.

27. The method of claim 26, characterized in that the excision is effected using the enzyme Cre recombinase.

28. The method of claim 27, characterized in that the Cre recombinase enzyme is administered as a recombinant fusion protein which can penetrate into cells.

29. The method of claim 27, characterized in that the cells are infected with a recombinant virus which expresses the Cre recombinase enzyme.

30. The method of anyone of claims 26 to 29, characterized in that the suicide gene is used to select for successful excision.

31. A cell which is prepared by the method of anyone of claims 10 to 30.

32. The use of the cells of claim 31 for preparing a transplant for regenerating an organ.

33. The use of the cells of claim 31 for producing a drug for treating chronic diseases.

34. A drug which comprises a therapeutically effective quantity of the cells of claim 31.

35. A plasmid which contains the gene complex of anyone of claims 1 to 6 and/or the gene complex of anyone of claims 7 to 9.

36. A viral vector which contains the gene complex of anyone of claims 1 to 6 and/or the gene complex of anyone of claims 7 to 9.

37. A transplant which contains the cells of claim 31.

38. The use of the cells of claim 31 for regenerating an organ.

39. A kit which comprises the gene complex of anyone of claims 1 to 6 and/or the gene complex of anyone of claims 7 to 9.
